(19) United States
(12) Patent Application Publication
(10) Pub. No.: US 2008/0119916 A1
(43) Pub. Date: May 22, 2008

(54) Cold Compress for Therapeutic Cooling

(76) Inventors: Ramsey Joe Choucair, Dallas, TX (US); Scott Emil Coleridge, Belle Mead, NJ (US); Janet Lynn Mariani, Dallas, TX (US)

Correspondence Address: Colin P. Cahoon;CARSTENS & CAHOON, LLP, P.O. Box 802334, Dallas, TX, 75380, US

(21) Appl. No.: 11/539,020

(22) Filed: Oct. 5, 2006

(51) Int. Cl. A61F 7/00

(52) U.S. Cl. 607/104; 607/96

(57) ABSTRACT

A novel cold compress designed for use as a cooling medium. The cold compress comprises a flexible bag having a plurality of spheres. Each sphere contains a heat transfer fluid. As the bag is cooled or frozen, each sphere and the fluid they contain become cool or frozen. The bag is then placed on a body pail of the patient who is recovering from surgery or injury. The small spheres allow maximum surface contact with the body part. This allows maximum and efficient heat transfer.

### BACKGROUND OF THE INVENTION

1. Technical Field

The present invention relates generally to a bag for use as a cooling medium. More specifically, the invention uses a bag filled with a plurality of fluid-filled spheres for use in therapeutic cooling.

2. Description of Related Art

Cold compresses are commonly used to provide cooling therapy to patients preparing for or recovering from trauma such as surgery or injury. Such cooling can reduce swelling in bodily tissues.

Ways to cool bodily tissue are known in the art. One such example is an ice pack. Ice is well suited as a cooling medium due to its large latent heat of fusion. Because ice has a large latent heat of fusion, it can absorb a relatively large amount of heat before it begins to melt. This property has led to the wide use of ice as a cooling means, especially therapeutic cooling. Some ice packs are thick and inflexible plastic containers filled with water that becomes ice upon freezing. Such packs, resembling a closed book, are flat and rigid. Another type of ice pack comprises a flexible rubber package with a screw on lid into which ice chunks can be placed. These ice packs, however, have great drawbacks. One drawback is that the ice pack is heavy. Another more serious drawback is that the ice pack presents a limited cooling surface area. This is especially true if the ice pack is being applied to a patient's face. The flat and rigid ice pack is incapable of conforming to the contours of a patient's face. The flexible rubber ice pack can better conform to contours than the flat ice pack, but the potential surface area is limited to the size of the ice chunks. Even if the ice is broken into smaller pieces, the resulting surface area is insufficient because as the ice melts, the water drains toward the low part of the pack forming a pool. When water drains into such pools, the cooling surface area is greatly reduced, and as a result the cooling efficiency is also reduced. Thus, the ice pack is unsuitable for some therapeutic purposes.

Prior art attempts to obviate the problem of limited surface area have instituted packs that exhibit flexibility. Some packs consist of water and an antifreeze agent. The antifreeze agent prohibits the pack from freezing and thus makes the pack flexible. However, because the water is not frozen, one drawback is that the unfrozen pack fails to take advantage of the large heat of fusion of the ice. Consequently, although the unfrozen pack is flexible, it fails to offer the same cooling potential as an ice pack. Other packs consist of a plurality of chambers, each chamber being filled with water. When the pack is frozen, the pack is bent and the ice is broken to provide limited flexibility. Although such a chambered pack takes advantage of the properties of ice, it also lacks the ability to contour a patient's face or other areas as it provides only limited surface area. Other packs consist of a polymer and water mixture that turns into a gel. One disadvantage in these packs is the propensity of water molecules to clump together and freeze. These clumps can be broken, but flexibility and surface area is still lost.

Despite all the options provided by the prior art, many medical professionals still use a frozen bag of peas as a therapeutic cooling medium. Because each pea acts independent of the other peas, when a bag of peas is placed on a patient's face the free-flowing peas are able to rise and fall to match the contours of the patient's face and/or other anatomical structures. This ability to mimic the contours of the patient's face maximizes surface contact and as a result provides efficient and effective cooling. Despite its wide use, there are several disadvantages of using peas. One such disadvantage is that organic matter decomposes and emits an odor because of bacterial contamination. Such decomposition and bacterial contamination can result in additional perceptive problems from transmission of odor through the bag material or as a result of leaking as bags of peas often have leaking seals. Such leaking can also result in medical problems as any bacteria can potentially undesirably leak onto the skin.

Another disadvantage is that medical professionals often place a bag or rag over the bag of peas for sanitary purposes. This reduces both the heat transfer and the contouring ability of a bag of peas. Additionally, over time a bag of peas becomes unusable. This results from many iterations of freezing and thawing of the vegetable which causes the peas to lose the ability to retain water. When a pea has lost the ability to retain water, it loses its integrity and becomes mushy. As a result, the bag of peas is essentially an ice pack, which exhibits many of the disadvantages of the ice pack discussed above. The water, not being absorbed by the pea, can form bridges or clumps of ice. This undesirably reduces surface contact. Thus, a need exists for a therapeutic cooling medium that mimics the high surface area and effectiveness of a bag of peas while further reducing disadvantages of the prior art. Further, a need exists for a therapeutic cooling medium that permits the addition of a bacteriostatic or bacteriocidal agent that inhibits or destroys the growth of bacteria.

### BRIEF SUMMARY OF THE INVENTION

The invention comprises a bag comprising a plurality of sealed spheres. The bag is flexible and conducts heat. The spheres can be made of a plastic or polymer or other suitable material having barrier properties. The sphere is water tight and comprises an outer non-stick material. The spheres contain water or other suitable heat transfer fluids.

In one embodiment, the spheres are made of sufficiently flexible material such that when the spheres are completely filled with the heat transfer fluid the spheres do not crack or split under the pressure but rather expand outward along with their contents.

In another aspect of the invention, the spheres are partially filled with fluid and thus comprise a void space. This void space permits expansion of the fluid when it is frozen.

In one embodiment, the cold compress exhibits high heat transfer, flexibility, and contour ability. The small spheres provide a high surface area to bag volume ratio. This high surface area and flexibility allows the bag to match the contours of a human body part, such as a face. In one embodiment, the outer surfaces of the spheres comprise non-stick properties to minimize clumping that would limit the surface area. Additionally, because of the barrier properties of the outer sphere surface, the spheres do not leak the fluid or absorb moisture. As a result, the spheres can be reused without decreasing efficiency. In one aspect, the fluid or heat transfer fluid comprises a bacteriostatic agent. In one embodiment, the fluid or heat transfer fluid comprises a bacteriocidal agent.

In one aspect, the present invention provides a method for providing a reusable cold compress as a cooling medium by providing a disposable sheath that the bag can be placed into. The above as well as additional features and advantages of the present invention will become apparent in the following written detailed description.

### BRIEF DESCRIPTION OF THE DRAWINGS

The novel features characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein:

FIG. 1 illustrates a top cut-away view of the sealed flexible bag in accordance with one embodiment of the present invention;

FIG. 2 illustrates a cross-sectional side view of a sphere in accordance with one embodiment of the present invention; and

FIG. 3 illustrates a side view of a sealed flexible bag depicted in FIG. 1 contouring to a patient's face; and

FIG. 4 illustrates a sheath that can be used to hold the flexible bag in accordance with one embodiment of the present invention

Like reference numerals represent equivalent parts throughout the several drawings.

### REFERENCE NUMERALS

100—Cold Compress
101—Bag seals
102—Bag
200—Sphere
202—Sphere outer diameter
204—Sphere inner diameter
206—Sphere outer layer
208—Heat transfer fluid
209—Void space
210—all thickness
300—Cold compress contouring to a patient's face
400—Sheath

### DETAILED DESCRIPTION OF THE INVENTION

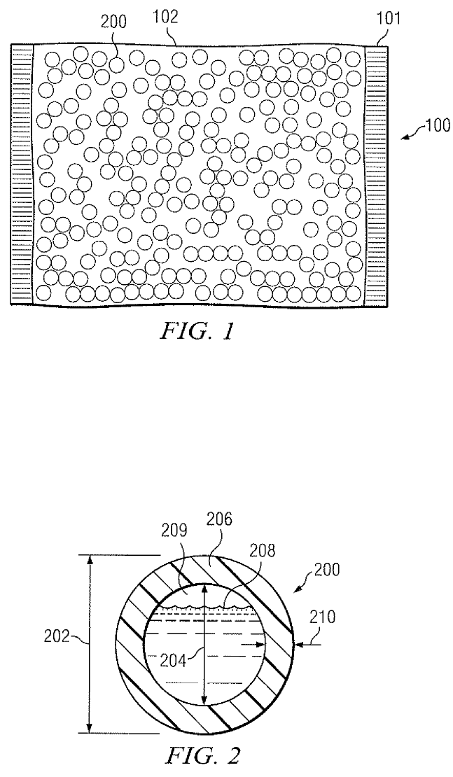

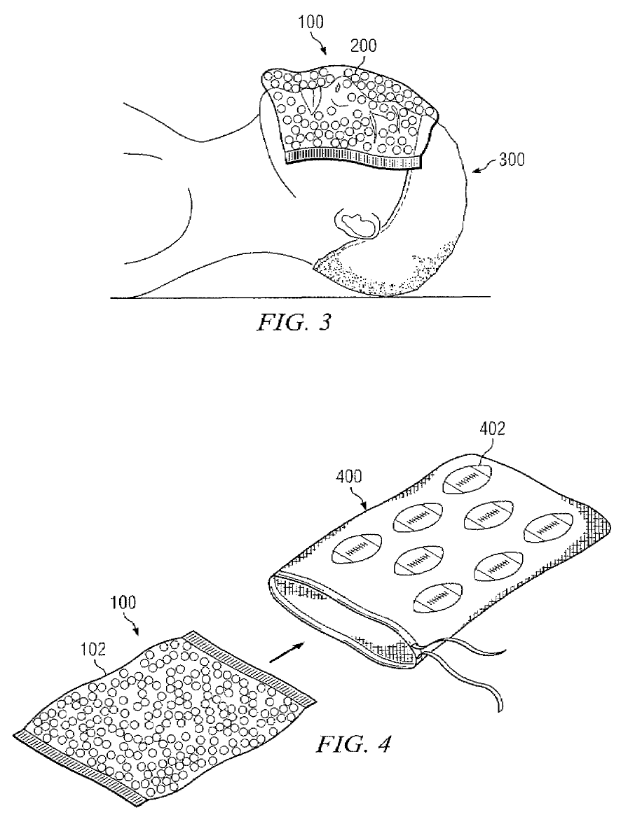

Referring now to the provided drawings, similar reference numerals represent the equivalent component throughout the several views of the drawings. FIG. 1 illustrates a top cut-away view of the sealed flexible bag in accordance with one embodiment of the present invention. FIG. 2 illustrates a cross-sectional side view of a sphere in accordance with one embodiment of the present invention. FIG. 3 illustrates a side view of a sealed flexible bag, depicted in FIG. 1, contouring to a patient's face.

Referring to FIG. 1, a cold compress 100 comprises a plurality of free-flowing spheres 200 inside the sealed bag 102. As used herein, free-flowing is used to describe an independent object that is not connected or attached to another object and that is free to flow and respond independently to pressure and gravity. Although the cold compress 100 is depicted as having two transverse seals 101 it can be made by methods known in the art such as with vertical form fill and seal machines or other suitable method. In one embodiment, the cold compress 100 comprises two sheets of overlapping film that is sealed along each of four sides. The sealed bag 102 can comprise any suitable sealed bag, regardless of the number of seals, and regardless of the method of manufacture. In one embodiment, the cold compress 100 can be seen to enclose the free flowing spheres 200.

In one embodiment, the cold compress 100 is capable of wrapping around itself and attaching to itself or another object by VELCRO, mechanical clips, tape, or any other suitable device so long as the cold compress 100 helps to fasten the bag 102 to an anatomical structure.

In one embodiment, the cold compress 100 comprises an affixing strap (not shown) that can be used to fasten the cold compress 100 to an anatomical structure. The affixing strap can wrap around a body member and affix to itself or to a portion of the cold compress 100. The size and shape of the cold compress 100 can be varied depending on the application. For example, if the cold compress 100 is to be applied to a patient's face, the bag 102 can have a slightly rectangular shape and be about the size of a sheet of paper (about 22 cm by 28 cm). Likewise, if an ankle is to be treated then the cold compress 100 could be long enough to wrap around the entire ankle yet wide enough to cover only the ankle.

The bag 102 can comprise any flexible material that conducts heat and preferably has barrier properties as to water vapor. A bag that has insulating properties (very low thermal conductivity) would decrease the effectiveness of the invention. The bag 102 is preferably made of a material that has barrier properties to water vapor. Barrier properties are preferred because if moisture was allowed to enter the bag, the formation of ice bridges and clumps across the bag can occur that can inhibit flexibility and contour ability because of decreased surface area. Additionally, undesirable moisture in the bag can bond to the spheres, and when frozen, form clumps of spheres. Consequently, in one embodiment, the bag 102 comprises sufficient barrier properties. As used herein, sufficient bag barrier properties is defined as a bag having a water vapor transmission rate of less than about 20 g/mill/645.16 sq. cm (100 sq. in.)/day at 37.8° C. (100° F.) and 90% relative humidity with a preferable rate of less than about 2 g/mill/645.16 sq. cm (100 sq. in.)/day at 37.8° C. (100° F.) and 90% relative humidity.

The material must also be flexible at temperatures lower than the freezing temperature of water (0° C.) as well as temperatures greater than about 38° C. (˜body temperature). The bag cannot become rigid and inflexible at lower temperatures as this will decrease the effectiveness of the bag. Likewise, the bag cannot melt or stretch at temperatures around body temperature (˜38° C.) because this is the common use temperature. In one embodiment of the invention, the bag 102 is made from flexible film known in the art. The flexible film can be made from polymers and polymer composites selected from high density polyethylene, medium density polyethylene, low-density polyethylene, polycarbonate, polypropylene, linear low density polyethylene, polyethylene terephthalate, polyvinyl chloride, polystyrene, polyurethane, polycarbonate or other suitable material.

In one embodiment, the bag 102 is made from other suitable materials or material composites, including, but not limited to, cloth-like materials such as micro-fibers, nylon, cotton, GORE-TEX, polyester blends, interwoven textiles and water-resistant paper such as waxy paper.

Referring to FIG. 2, the sphere 200 has an outer diameter 202 and an inner diameter 204. In one embodiment, the outer diameter 202 ranges from between about 2 mm to about 25 mm, more preferably between about 3 mm to about 10 mm, and with a most preferred diameter of about 8 mm. The inner diameter 204 is a function of the thickness 210 of the sphere outer layer 206. The inner diameter 204 is equal to the outer diameter 202 less twice the wall thickness 210 of the sphere 200. Any suitable wall thickness 210 can be used, however; thinner wall thickness is preferred as it allows for increased heat transfer. Different values for inner 204 and outer diameters 202 can be selected depending on the desired size of the sphere 200. Smaller spheres yield greater surface area but require a greater manufacturing cost.

The sphere outer layer 206 can be made of any material that has sufficient barrier and heat transfer properties. The sphere outer layer 206 must conduct heat, must be water tight, and must be sealed to prevent leaks. The sphere outer layer 206 should act as a sufficient barrier to water vapor. As used herein, sufficient sphere barrier properties is defined as a sphere outer layer 206 having a water vapor transmission rate of less than about 20 g/mill/645.16 sq. cm (100 sq. in.)/day at 37.8° C. (100° F.) and 90% relative humidity with a preferable rate of less than about 2 g/mill/645.16 sq. cm (100 sq. in.)/day at 37.8° C. (100° F.) and 90% relative humidity. The sphere outer layer 206 preferably comprises a low coefficient of expansion. The reason for this is that if the outer layer 206 shrinks or expands with temperature at a much different rate than the fluid 208 it contains, then the sphere could crack or split. Finally, in one embodiment, the sphere outer layer 206 comprises a non-stick material. As used herein, a non-stick material is one that does not bond or stick to itself so that the spheres remain free-flowing. If the sphere outer layer 206 was made of such a material (e.g., a gel) then the spheres would tend to cluster, reducing surface area and heat transfer. In one embodiment of the current invention, the outer sphere layer 206 is selected from one or more polymer or composites selected from, low, medium, or high density polyethylene, polypropylene, linear low density polyethylene, polyethylene terephthalate, polyvinyl chloride, polystyrene, polyurethane, or other suitable material. Other suitable non-stick, water tight materials can include, but are not limited to, metals, or metal composites, such as aluminum or steel, or a silica-based material such as fiberglass.

Alternatively, in one embodiment, the outer sphere layer 206 is coated with a non-stick material such as a fluoropolymer such as polytetrafluoroethylene (PTFE) or a silicone-based coating comprising silicone resins, elastomers, oils or silicone glazes to help ensure the spheres are free-flowing.

In one embodiment, the sphere 200 is partially filled with a fluid 208 that is liquid at ambient conditions. Because of convenience and the high latent heat of fusion of H₂O, in one embodiment of the invention the fluid 208 comprises water. The water can be then be frozen into ice. Using ice permits the user of the instant invention to absorb a large amount of heat before the ice begins to melt. Additionally, because of the availability of freezers, the temperature required to make ice, 0° C., is very reachable and convenient.

The conduction of heat is expressed by the mathematical formula below:

Q=cmΔT

Where Q=Heat Conducted or Heat Transferred
c=specific heat of the material conducting the heat
m=mass of the substance conducting the heat
ΔT=the temperature difference between the two mediums where heat transfer is taking place.

A material's heat capacity, denoted as “c” in the equation above, is quantified by amount energy required to raise the temperature of the material by a certain amount. In one embodiment, a fluid having a relatively high heat capacity is used. The fluid 208 should have a high specific heat, greater than about 1 J/gram/Kelvin at 25° C. (constant pressure), and preferably greater than about 4 J/gram/Kelvin at 25° C. (constant pressure) in the liquid phase. Water in the liquid form exhibits a higher specific heat or heat capacity than water in the solid form (ice). For example, the heat capacity of water is 4.187 kJ/kg K and the heat capacity of ice is 2.108 kJ/kg K. Accordingly, faster heat transfer may be possible if liquid water is utilized. One advantage of the present invention is that, because the fluid 208 is placed into water-tight spheres, the fluid 208 can comprise coolants such as gels that would otherwise be undesirable because of the tendency of such gels to stick to one another. Thus, a fluid can be selected to maximize the amount of cooling time provided by the cold compress 100 of the present invention.

The driving force, denoted as “ΔT” in the equation above, is the temperature difference between the temperature of a patient's body part and the temperature of the bag material in communication with the body part. The driving force can be increased by supplying a colder medium in communication with the body part. One simple way to accomplish this is to add salt or an antifreeze solution to the water solution, thus lowering the freezing point. Depending on the concentration of the salt-water mixture, temperatures as low as −21° C. (for NaCl) can be reached without the mixture freezing. Further, because the spheres are water tight, and are further in a sealed flexible bag, any fluid used is less likely to leak and come into contact with a user than many prior art embodiments. Consequently, heat transfer fluids that may otherwise not be advisable for use can be used in accordance with various embodiments of the present invention.

As further clarification, one objective of one embodiment of the present invention is to provide a cold compress that can transfer at least as much heat (Q) from an anatomical member as a similar sized bag of frozen peas. As shown by the above formula, this can be achieved by using heat transfer fluids with high “c” values and/or by increasing the driving force (ΔT).

Consequently, in one embodiment, the driving force is maximized by using a heat transfer fluid that does not freeze in a standard freezer, e.g, at temperatures lower than −22° C. Thus, in one embodiment, the heat transfer fluid comprises an antifreeze solution. This can permit a larger driving force to be utilized.

Alternatively, in one embodiment, it may be desirable to take advantage of the latent heat of fusion provided by a frozen fluid and a larger driving force than would be provided by water alone. Thus, in one embodiment, the heat transfer fluid comprises a chemical mixture to decrease the freezing point of the heat transfer fluid, but that permits the heat transfer fluid to freeze in a standard freezer, e.g., at temperatures higher than −22° C. The chemical mixture can be a salt (MgCl, NaCl, etc) added to water or any other suitable mixture that results in a freezing point of higher than −22° C.

The volume of fluid 208 to be filled in each sphere 200 depends on the expansion coefficient of the fluid 208 and the outer sphere layer material 206. Determination of the amount of fluid 208 to fill a given sphere size is within the knowledge of one skilled in the art. In one embodiment the sphere 200 is only partially filled with liquid 208 at room temperature. When partially filled, the sphere 200 has sufficient void space 209 within it to allow for liquid expansion at reduced temperatures. This will prevent the sphere from cracking or splitting due to the expansion pressure of the fluid 208. For example, it is known that water expands in volume by about 10% when frozen. As a result, in one embodiment the sphere 200 has a void space that occupies about 10% or more of the volume of the water. Depending on the expansion coefficient of the fluid 208 and the material used in manufacturing the sphere outer layer 206 the void space 209 can occupy up to about 50% of the total sphere volume, with a preferred void space volume of less than about 25% and most preferably between about 5% and about 15% of the total sphere volume as calculated by the sphere inner diameter 204. This allows the water to expand when frozen, and not split or crack the outer sphere layer 206. In another embodiment, the sphere 200 is completely filled with fluid. In such embodiment, the sphere outer layer 206 in this embodiment is made of a flexible material that can expand with the fluid 208. This flexible material allows the fluid to expand without causing the sphere to crack or split. In one embodiment, the sphere thickness 210 is adjusted according to the level of fluid placed into the sphere 200.

In one embodiment, the spheres 200 can be manufactured on a machine called a “Blow-fill-seal Machine.” Such machines are known in the art and commonly used for the aseptic packaging of pharmaceuticals. The spheres can be made as follows: First, the outer sphere layer 206 is formed by extruding the material around a mold and blowing air into the mold to form the bulk of the sphere 200. Next, a measured dose of fluid 208 is injected into the partial sphere 200. Finally, the sphere 200 is capped or sealed on top.

Other suitable machines or methods can be used to manufacture the spheres 200. For example, U.S. Pat. Nos. 5,254,379 and 6,532,947 disclose methods of filling a substantially spherical object with a fluid. While these patents are directed towards paintballs, those skilled in the art will understand that such methods can be adapted to make the spheres of the present invention. Other suitable machines or methods such as vacuum form molding can also be used.

Referring to FIG. 3, the cold compress or therapeutic bag 100 is shown contouring a patient's face. Given that the spheres are small in size, the surface area and therefore surface contact between the cold compress 100 and the patient's face is maximized. Additionally, because sphere outer layer 206 is made from a non-stick material, clumps and clusters are avoided. This allows individual, free-flowing spheres to rise and fall with the contours of, for example, a human face. Thus, in operation, the instant invention mimics the successful utilization of a bag of peas. However, the current invention, unlike a bag of peas, can be reused many times without decreased efficiency by simply re-freezing the bag. Further, undesirable odors are avoided that can occur from the chemical breakdown of peas. Additionally, in one embodiment, the instant invention has a strap or other applicable device that secures the bag 102 in place. This eliminates the need for the patient to hold the bag in place.

In one embodiment, the cold compress 100 is placed into a sheath that is sized such that the cold compress 100 can be placed inside the sheath. FIG. 4 illustrates a sheath 400 that can be used to hold the flexible bag in accordance with one embodiment of the present invention. The sheath 400 can be a configured in any suitable manner and its configuration can emulate a pillowcase, a sock, a sock-like sheath having a drawstring, etc. The sheath 400 can be of any suitable material such as those disclosed above as suitable for the bag 102. In one embodiment, the sheath comprises a decorative design 402. For example, the decorative design 402 can include a picture of a football and may be popular for application of the cold compress 100 to sports injuries (e.g., an ankle injury). In one embodiment, the sheath 400 comprises a material that is conducive to the clientele paying for a high end surgical operation. Consequently, in one embodiment, the present invention provides a method for providing a cold compress 100 for therapeutic cooling that is reusable by health care providers. For example, in one embodiment, before the cold compress 100 of the present invention is used on a patient, a sheath 400 is applied to the outer portion of the bag 102. When the cold compress 100 is subsequently removed, the sheath can be removed and discarded and the cold compress 100 can be optionally washed and/or disinfected and placed into a freezer for re-use. The sheath can also be made from many of the same materials disclosed above to make the bag 102.

In one embodiment, the sheath comprises a device for attaching the bag 102 to an anatomical structure. The same devices discussed above, such as VELCRO, mechanical clips, tape, or any other suitable device so long as the cold compress 100 helps to fasten the bag 102 to an anatomical structure.

While the invention has been described with respect to a preferred embodiment, other embodiments are possible as one of ordinary skill in the art will recognize that one can modify the particulars of the embodiment without straying from the inventive concept.

We claim:

1. A cold compress used for therapeutic cooling comprising:
a sealed flexible bag comprising a plurality of free-flowing spheres, wherein said sealed flexible bag contains a plurality of free-flowing spheres, wherein further each of said plurality of free-flowing spheres has an inner diameter and an outer diameter, wherein further each of said plurality of free-flowing spheres has an outer layer and is at least partially filled with a heat transfer fluid having a freezing point to create a plurality of at least partially filled free-flowing spheres with said sealed flexible bag, wherein further said outer sphere layer comprises sufficient sphere barrier properties to water vapor.

2. The cold compress of claim 1, wherein said bag further comprises a strap used to attach said bag to an anatomical structure.

3. The cold compress of claim 1, wherein the heat transfer fluid comprises water.

4. The cold compress of claim 1, wherein the heat transfer fluid comprises a chemical mixture added to decrease the freezing point of said heat transfer fluid.

5. The cold compress of claim 1, wherein the heat transfer fluid comprises water with an antifreeze solution added so as to decrease the freezing point of said heat transfer fluid.

6. The cold compress of claim 1, wherein the heat transfer fluid comprises a bacteriostatic agent.

7. The cold compress of claim 1, wherein the heat transfer fluid comprises a bacteriocidal agent.

8. The cold compress of claim 1, wherein said outer diameter is between about 3 and about 10 mm.

9. The cold compress of claim 1, wherein each of said partially filled free-flowing spheres comprises a void space.

10. The cold compress of claim 1, wherein said outer layer of said free-flowing spheres comprises a non-stick coating.

11. The cold compress of claim 1, wherein said outer layer of said free-flowing spheres comprises one or more polymers selected from low-density polyethylene, medium density polyethylene, high density polyethylene, polypropylene, linear low density polyethylene, polyethylene terephthalate, polyvinyl chloride, polyurethane, polycarbonate and polystyrene.

12. The cold compress of claim 1, wherein said flexible bag comprises a material selected from one or more polymers selected from low-density polyethylene, medium density polyethylene, high density polyethylene, polypropylene, linear low density polyethylene, polyethylene terephthalate, polyvinyl chloride, polyurethane, polycarbonate, and polystyrene.

13. The cold compress of claim 1, wherein said free-flowing spheres are manufactured by a blow-fill-seal machine.

14. The cold compress of claim 1, wherein said flexible bag comprises a material having sufficient bag barrier properties to water vapor.

15. The cold compress of claim 1, wherein said outer sphere layer can expand with said fluid when said fluid is frozen so as not to crack.

16. The cold compress of claim 1, wherein said free-flowing spheres are made with a non-stick material.

17. A method for providing a re-usable cold compress as a cooling medium, comprising the steps of:
a) providing the cold compress of claim 1; and
b) providing a sheath sized such that said cold compress can be placed inside said sheath.

18. The method of claim 17 wherein said sheath comprises a decorative sheath.

19. The method of claim 17 wherein said sheath comprises a material selected from one or more polymers selected from low-density polyethylene, medium density polyethylene, high density polyethylene, polypropylene, linear low density polyethylene, polyethylene terephthalate, polyvinyl chloride, polyurethane, polycarbonate, and polystyrene.

20. The method of claim 17 wherein said sheath comprises a material selected from one or more cloth-like materials.
